(19) United States
(12) Patent Application Publication
(10) Pub. No.: US 2013/0177630 A1
(43) Pub. Date: Jul. 11, 2013

(54) ANTI-CANCER COMPOSITION AND METHOD UTILIZING 3-BP AND LIPOSOMAL REDUCED GLUTATHIONE

(71) Applicant: Frederick Timothy Guilford, Palo Alto, CA (US)

(72) Inventor: Frederick Timothy Guilford, Palo Alto, CA (US)

(73) Assignee: Frederick Tmothy Guilford, Palo Alto, CA (US)

(21) Appl. No.: 13/734,742

(22) Filed: Jan. 4, 2013

Related U.S. Application Data
(60) Provisional application No. 61/748,619, filed on Jan. 3, 2013.
(60) Provisional application No. 61/647,707, filed on May 16, 2012.
(60) Provisional application No. 61/645,572, filed on May 10, 2012.
(60) Provisional application No. 61/583,388, filed on Jan. 5, 2012.

(51) Int. Cl. A61K 38/06

(52) U.S. Cl. CPC A61K 38/063
USPC 424/450; 514/19.3

(57) ABSTRACT

The invention proposes a method of treatment of cancers exhibiting a CD163 characteristic by a liposomally formulated reduced glutathione.

The invention proposes a method of treatment of cancer of a combination of liposomally formulated reduced glutathione to cooperate with an anti-cancer agent 3-bromopyruvate and its analogs Bromopyruvic acid and 3-BrOP (3-bromo-2-oxopropionate-1-propyl ester) (collectively “3BP” or “3-BP”) to ameliorate the 3BP side effects and enhance its cancer-killing capacity and enhance the detoxification of the body of dead cancer cell debris.

### PRIORITY AND CONTINUATION DATA

This application claims benefit of and priority to U.S. Prov'l Appl. No. 61/61/583,388 filed Jan.5, 2012, U.S. Prov'l Appl. No. 61/645,572 filed May 10, 2012, and U.S. Prov'l Appl. No. 61/647,707 filed May 16, 2012, and a provisional application of this name filed Jan. 3, 2013 with U.S. Prov'l Application No. 61/748,619, and if needed for any national or regional stage is a continuation of those applications, each of which applications is incorporated by reference as if fully stated in this application.

### FIELD OF INVENTION

The invention relates to the treatment of cancer by novel method using a novel composition.

### SUMMARY

The invention proposes a method of treatment of cancers exhibiting a CD163 characteristic by a liposomally formulated reduced glutathione.

The invention proposes a method of treatment of cancer of a combination of liposomally formulated reduced glutathione to cooperate with an anti-cancer agent 3-bromopyruvate and its analogs Bromopyruvic acid and 3-BrOP (3-bromo-2-oxopropionate-1-propyl ester) (collectively “3BP” or “3-BP”) to ameliorate the 3BP side effects and enhance its cancer-killing capacity and enhance the detoxification of the body of dead cancer cell debris.

### BACKGROUND

Macrophages (MP's) play a significant role in the management of infected or damaged tissues. In cancers, tumor-associated macrophages (TAMs) have been shown to constitute a significant part of the tumor-infiltrating immune cells. Investigation of the MP's in tumors show them to be divided into two general groups based on the expression of cytokines by the MP's and described as M1 or M2.

The classification as to M1 or M2 is determined by the expression of Interleukins, a group of cytokines (secreted proteins/signaling molecules) that are released by leukocytes (white blood cells) and act on leukocytes. A phenotype (from Greek phainein, ‘to show’ +typos, ‘type’) is the composite of an organism's observable characteristics or traits. Phenotypes result from the expression of an organism's genes as well as the influence of environmental factors and the interactions between the two.

Classical macrophages, noted as Ml, have been characterized as a phenotype characterized by interleukin 12—High (IL-12^{high}) Interleukin 23—High (IL-23^{high}), and interleukin 10—low (IL-10^{low}). M1 macrophages are immune effector cells that are aggressive against microbes and can engulf and digest affected cells much more readily, M1 macrophages produce reactive oxygen and nitrogen intermediates as well as inflammatory cytokines and play a role in upregulating T helper cell 1 (Th1) responses are mediated by the white blood cells that help other immune cells by activating and directing their function. They help maximize the bactericidal activity of phagocytes such as macrophages. TH1 activity functions in a manner that continues an efficient and effect macrophage cell function in terms of killing invaders such as infection, parasites and cancer cells (1).

The M2 macrophage phenotype is characterized by an IL-12¹″, IL-23¹″, and IL-10^{high} presentation IL-10 is involved in turning off immune system activation and helps decrease inflammation. The function of M2 macrophages is diverse, but in general they are involved in T helper 2 (Th2) response, whose main partners are B-cells which is generally associated with the production of antibodies from B-cells. M2 type macrophages have an immunoregulatory function, and orchestrate encapsulation and containment of parasites and promote tissue repair, remodeling, and tumor progression (1).

| Macrophage Type | L-12 | IL-23 | IL-10 |
|---|---|---|---|
| M1 | High | High | Low |
| M2 | Low | Low | High |

An immunological marker distinguishing macrophages from other immune cells is the marker CD68. In the immune system the type of white blood cell called lymphocytes have been found to perform different functions in immune defense. Before the function of these cells was understood, a way to identify the cells was found using antibodies specific to various clusters of proteins found on the surface of the lymphocyte. These antibodies were able to chart the different types of lymphocyte populations based on the appearance of specific immunologically distinctive protein clusters as markers. These protein markers ultimately were associated with functionally distinct populations of lymphocytes such as B-cells, helper T-cells (TH), cytotoxic T-cells (TC), and natural killer (NK) cells. These different populations have become designated by the cluster of differentiation (CD) antigen number. The first group identified was CD group 1, designated CD1. The second was designated CD2 and so on. At the time this designation was being formed, the actual function of the lymphocytes was not known. It has been subsequently shown that the white blood cells, called T helper (TH) lymphocytes always show a cluster designation number 4 and are now known as CD4. The cluster of differentiation (CD) CD68 is associated with macrophages and the presence of this marker makes it useful in diagnosing the accumulation of macrophages in various tissues.

Macrophages, from the Greek, meaning “large eaters,” are large phagocytic leukocytes, which are able to move outside of the vascular system by moving across the cell membrane of capillary vessels and entering the areas between cells in pursuit of invading pathogens. In tissues, organ-specific macrophages are differentiated from phagocytic cells present in the blood called monocytes. Macrophages are the most efficient phagocytes, and can phagocytose substantial numbers of bacteria or other cells or microbes. The binding of bacterial molecules to receptors on the surface of a macrophage triggers it to engulf and destroy the bacteria through the generation of a “respiratory burst”, causing the release of reactive oxygen species. Pathogens also stimulate the macrophage to produce chemokines, which summons other cells to the site of infection.

In cancer, macrophage infiltration around a tumor may help delay tumor development. This suggests that peritumoral tumor-associated macrophages (TAM) are associated with increased survival of the host and a better prognosis in tumors such as colon cancer. This suggests that peritumoral macrophages are of the M1 phenotype. In contrast, intratumoral TAM count has been correlated with depth of invasion, lymph node metastasis, and staging of colon and rectal cancers, suggesting that intratumoral M2 macrophages cause cancer cells to have a more aggressive behavior (1).

It has been suggested that these contradictory functions of MP's may have different additional markers. While CD68 is a general marker of MP's the use of subset markers such as CD163 or CD204 might have an increased significance. The use of CD204 as a marker of macrophages in lung adenocarcinoma has a strong association with poor outcome (2). In a similar fashion CD164+TAM has been shown to correlate with myometrial invasion in endometrial carcinoma of the uterus (3). In pancreatic cancer, high numbers of CD163- or CD204-positive macrophages were associated with poor prognosis (P =0.0171); however, this was not the case for the number of CD68-positive macrophages (4).

CD 163, a haptoglobin-hemoglobin complex is implicated as a hemoglobin scavenger receptor for binding of erythrocytes to macrophages for the removal of iron containing proteins and is expressed in monocytes and macrophages. CD163 can also function as a macrophage receptor for both Gram-positive and -negative bacteria (5). Recent work has shown that this marker is also specific for neoplasms of histiocytic differentiation in the skin (6).

The inventor has hypothesized that the receptor for CD163 may preferentially attach to the liposome of the liposomal reduced glutathione in a manner similar to the absorption demonstrated by the macrophages from individuals with HIV that are undergoing infection (Unpublished data Venketaraman and Guilford, Western University 2012). The presence of CD163 appears to increase the absorption of the liposome and its glutathione content. The result of this surprising absorption of glutathione using liposomal reduced glutathione correlates clinically to the surprising and unexpected finding of resolution of the Merkel Cell Carcinoma reported in the Case Example. The invention also proposes using PEG headed thermodynamically stable liposomes made in a thermodynamically stable environment, but the lecithin based formulation may be more effective that the PEG headed formulation.

The use of liposomal reduced glutathione is referenced for use in not only in the skin tumor described above, but also in melanoma Melanomas with dense CD163(+) macrophage infiltration in tumor stroma and CD68(+) macrophage infiltration at the invasive front that are associated with poor overall survival (7). The marker CD163 has also been identified in other tumor cells such as breast cancer tumors (8) and rectal tumor. When the [need antecedent and identification of antecedent for “this”]CD163 phenotypic macrophage scavenger receptor trait [is found in cancer tissues, it is related to early local recurrence, shorter survival time and reduced apoptosis (9). Cancers of a more advanced histological grade correlating with more aggressive tumors and decreased host survival express CD163 to a higher extent (8).

For example, the presence of macrophages expressing CD163 infiltrating Hodgkin's lymphoma correlate with and increase in Epstein-Ban virus laden cells and a worse prognosis (10). Additionally, High CD163-positive macrophage levels significantly correlated (P=0.007) with a poor outcome in patients with oral squamous cell cancer (OSCC) (11).

Studies have shown that NAC, a form of cysteine, which can be the rate limiting factor in the formation of the tripeptide glutathione (γ-glutamyl cysteinyl-glycine) will restore glutathione levels in depleted lymphocytes and stimulate the cell mediated response known as TH1 (17). Cytokines are small protein-like molecules called polypeptides that are secreted from monocytes and lymphocytes after interaction with a variety of materials such as antigens, toxins or even other cytokines. As they circulate locally as well as systemically through the blood they function like immune hormones. Cytokines affect the magnitude of inflammation or immune responses. While they can be released by lymphocyte interaction with a specific antigen, they can be released by non-specific antigens. Thus cytokines bridge both the innate and adaptive immune systems. The type of response to immune challenge is determined by the cytokines that are released during the challenge. The T cells called helper cells determine this response based on the cytokines that they release. For the purpose of description of activity the response stimulated by the TH cells is referred to as being of two types, TH1 and TH2. The TH1 pattern is characterized by the release of interleukin-12 (IL-12) and interferon γ (IFN-γ) production. These cytokines increase the cell-mediated immunity. The TH2 response characterized by IL-4 and IL-10 production and the up-regulation of the production of antibodies such as Immunoglobulins G and E (IgG and IgE). The cytokines related to the two different responses tend to each down regulate the other. For example IFN-γ inhibits TH2 associated cytokine production and IL's 4 and 10 inhibit TH1 associated function. When the balance between TH1 and TH2 responses reaches an extreme the ability to overcome infection either locally or through the whole body is impaired (Peterson) (17). IFN-γ will also inhibit TH2 associated cytokine and decrease IL-10.

The macrophages from individuals with human immunodeficiency virus (HIV) have been shown to be low in glutathione and particularly vulnerable to infection with Mycobacterium tuberculosis (the infectious agent of the disease known as Tuberculosis). An additional unpublished study shows that liposomal reduced glutathione formulated per this invention has a significantly increased absorption and function in the macrophages from individuals with HIV that are undergoing infection with M. tb. The absorption of the liposomal glutathione is 1000×'s more efficient than the glutathione precursor N-acetyl cysteine (NAC) in restoring normal glutathione levels and restoring the glutathione related function of slowing the replication of M tb in macrophages taken from individuals with HIV . . . “Glutathione Supplementation Improves Immune Function in HIV+Macrophages,” Morris D, Guerra C, Khurasany M, Guilford T, Venketaraman V, (unpublished, Western University of Health Sciences, Pomona, Calif. 91766, USA) (“Morris D”).

Merkel Cell Carcinoma (MCC) is a rare skin cancer that often harbors Merkel cell polyomavirus (MCPyV) DNA (12). The incidence of MCC is increasing (13). MCC is highly malignant, of neuroendocrine origin and while presenting on the skin it is characterized by frequent lymphatic metastasis. Metastasis can occur both local and regional (13). Lymphovascular invasion (LVI) is a predictor of recurrence (30%) or death (15%) from MCC, when found in regional lymph nodes (15), while the median survival of 26 patients was 29 months increasing (13). A number of immune cells are associated with MCC including T-lymphocytes (CD3-positive cells), T cell subsets (CD4, CD8, and FoxP3-positive cells), natural killer cells (small CD16-positive cells), and macrophages (CD68 and CD163-positive cells)(12). The presence of [define VEGF] VEGF-C(+)CD68(+) CD163(+) suggests that an M2 type of macrophage infiltrate occurs in MCC and is associated with lymphangiogenesis (increased new blood vessel formation and metastasis in MCC (14). The presence of the CD163 marker on macrophages infiltrating Merkel cell tumors appears to be a typical finding of Merkel Cell Cancer and appears to contribute to the aggressive nature of this cancer.

It has been shown that liposomal reduced glutathione can increase the production of IL-12 in macrophages undergoing infection with M. tb. (Venketaraman and Guilford 2012 (not yet published). During the treatment of Merkel Cell carcinoma, it appears that liposomal reduced glutathione can stimulate the production of IL-12. This may be due to the interaction of glutathione in the presence of IFN-γ, which may stimulate the production of IL-12. The combination results in converting the tumor associated macrophages from an M2 phenotype to macrophages with M1 phenotype responses, results in the macrophages becoming cancer-killing cells. The presence of the M1 type macrophages would then result in the surprising effect of eliminating the metastatic lesions from the individual in the case report.

The surprising and novel finding in the unpublished Morris D et al study of the dramatic absorption of liposomal reduced glutathione compared to N-acetyl cysteine (“NAC”) explains the ability of this formulated form of liposomal reduced glutathione to restore macrophage function back to the M1 function.

“In a previous study we observed elevated levels of TGF-β in both the plasma and macrophage culture supernatants of HIV+macrophages [42]. This elevated TGF-β will compromise the amount of GCLC present inside the cell; consequently, supplementing the raw materials [such as with NAC] for de novo synthesis in HIV+individuals who are over expressing TGF-β will not result in the same increased production of reduced GSH that is observed in individuals who are not over expressing TGF-β. In addition, this phenomenon may explain why 1GSH [the liposomal reduced glutathione of this invention] at lower concentrations than NAC is more effective at raising the concentration of reduced GSH in HIV+macrophages than in HIV−macrophages. Supplementing with an 1GSH formulation provides complete GSH molecules to cells, circumventing the enzymatic pathway responsible for GSH production, without the requirement for the cell to construct the tripeptide [43]. This may also explain why treatment with 1GSH seems to raise the ratio of reduced GSH to GSSG at much lower concentrations than NAC, as cells treated with NAC will have to produce new molecules of reduced GSH utilizing their own enzymatic machinery. [emphasis added].” Morris et al at pp. 17-18.

It has long been conjectured that administration of glutathione to cancer cells would increase the function of cancer cells, contrary to the teaching of this invention. This invention is based on the contrary proposition that liposomal reduced glutathione is able to decrease the viability of tumors such as Merkel cell carcinoma which are bearing the designation CD163. The lecithin used in the formation of liposomal reduced glutathione, particularly the use of hydroxylated lecithin, may facilitate the attraction of the liposomal reduced glutathione particle to the CD163 bearing cells and is considerably more potent than NAC.

Recently, in an article entitled “A Translational Study “Case Report” on the Small Molecule “Energy Blocker” 3-Bromopyruvate (3BP) as a Potent Anticancer Agent: From Bench Side To Bedside, Ko Y H, Verhoeven H A, Lee M J, Corbin D J, Vogl T J, Pedersen P L, J Bioenerg Biomembr. 2012 February; 44(1):163-70. Epub 2012 Feb. 11, PMID:22328020, treatment of fibrolamellar hepatocellular carcinoma by 3-bromopyruvate was reported with life-extension characteristics, but severe side effects. 3BP is said to be a “potent and specific anticancer agent . . . different in its action from most currently available chemo-drugs . . . [by targeting] cancer cells' energy metabolism, both its high glycolysis (‘Warburg’) effect and mitochondrial oxidative phosphorylation . . . [thereby inhibiting/blocking] total energy production leading to a depletion of energy reserves.” While first presented in 2000, Ko et al. (2001) Cancer Lett. 173, 83-91”[fill in PMID] and further discussed as a “highly effective and rapid anticancer agent in vivo in 2004 (Ko et al. (2004) Biochem. Biophys. Res. Commun. 324: 269-75, its adoption has been slow because of side effects. This invention proposes to attenuate the side effects while simultaneously potentiating the characteristics of 3BP.

Case Report

A 92 year old man initially presented with Merkel Cell Carcinoma presenting initially on the left forearm in November 2010. Excisional biopsy showed Merkel Cell Carcinoma of the skin. Subsequent PET scan showed an axillary lymph node metastasis, which was treated with radiation. Subsequent scans showed metastasis to the liver, again treated with radiation.

During evaluation for a cough, follow up scan in February 2012 showed cancer metastasis in the left kidney and stomach. The individual began using 1 teaspoon liposomal reduced glutathione per day.

A follow up scan on Apr. 9, 2012 in preparation for additional radiation treatment showed resolution of the kidney and stomach lesions. As the CD163 is associated with M2, tumor enhancing functions of macrophages, it is referenced in the current application that liposomal reduced glutathione will also restore M1 tumor killing function of TAM of a variety of tumors such as the lung adenocarcinoma, endometrial cancer and pancreatic cancers that were found to have M2, CD163+macrophages associated with the tumor as described above.

### PREFERRED MODE OF INVENTION.

Preferred Dosing

For systemic adjunctive management of cancer and support of anti-cancer immune function in individuals with cancer

Oral liposomal glutathione 1.5 (approximately 600 mg) teaspoons twice a day. More consistent dosing and effect occurs on an empty stomach but that is not essential to method of the invention.

For skin cancer such as Merkel Cell Carcinoma or melanoma

The oral formulation (8.25% w/w) can be applied topically to the area of the abnormal skin tissue. The formulation can be brought to 100% w/w by addition of deionized water.

Preferred Topical formulation of 5% (w/w) liposomal glutathione in a formulation sold as a Qusome by Biozone Laboratories, Pittsburgh, Calif. Applied two times per day to site of abnormal tissue

Formulation

Glutathione can be obtained from various sources including Kyowa Hakko U.S.A., 85 Enterprise, Suite 430, Aliso Vieja, Calif. 92656.

### EXAMPLE 1

Liposomal glutathione Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage | |
| retardant) | |
| Glutathione | 8.25 |
| (reduced) | |

A lipid mixture having components lecithin, ethyl alcohol and glycerin were commingled in a large volume flask and set aside for compounding. Hydroxylated lecithin is the preferred ingredient.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stifling plate, covered with parafilm and mixed with a magnetic stir bar until cooled to room temperature. Normally, citrus seed extract or flavorant would be added for taste enhancement. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al U.S. Pat. No. 5,891,465, U.S. Pat. No. 6,958,160 and U.S. Pat. No. 7,150,883 are incorporated in this description.

Concentrations of liposomal glutathione from 3.3%, 4%, 5%, 6%, 7%, 7.5%, 8%, 8.5% or 9% liposomal glutathione may be formed and utilized for dosing by decreasing the amounts of glutathione and preplacing the material with an increase in the sterile water concentration. The amount of 3.3% w/w Glutathione (reduced) is 123 mM in concentration in the liposomes.

### EXAMPLE 2

Liposomal glutathione Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage | |
| retardant) | |
| Glutathione | 8.50 |
| (reduced) | |

A lipid mixture having components lecithin, ethyl alcohol and glycerin were commingled in a large volume flask and set aside for compounding.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stifling plate, covered with parafilm and mixed with a magnetic stir bar until cooled to room temperature. Normally, citrus seed extract would be added. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat No. 5,891,465, U.S. Pat. No. 6,958,160 and U.S. Pat. No. 7,150,883 are incorporated in this description.

Concentrations of liposomal glutathione from 3.3%, 4%, 5%, 6%, 7%, 7.5%, 8%, 8.5% or 9% reduced glutathione may be formed and utilized for dosing by decreasing the amounts of glutathione and replacing the material with an increase in the sterile water concentration. The amount 3.3% w/w Glutathione (reduced) is 123 mM in concentration in the liposomes.

### FURTHER EXAMPLES 3

Formulation for Topical Application of Liposomal Reduced Glutathione

A topical cream or lotion containing reduced glutathione in a self-forming liposome sold under the brand name “QuSome” ® by Biozone Laboratories, Inc. of Pittsburgh, Calif. is another preferred embodiment. The Qusome self-forming liposome can be formed containing reduced liposomal glutathione in a concentration of 5% reduced glutathione in the liposome. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. Pat. No. 6,958,160 and U.S. Pat. No. 7,150,883 are incorporated in this description. The most important details of that manufacturing are as follows:

The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In formulating the liposomes, In one aspect, the invention includes a method of preparing liposomes. The method comprises providing an aqueous solution; providing a lipid solution, where the solution has a Pₐ between about 0.84 and 0.88, a Pᵥ between about 0.88 and 0.93, and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolyglycerol-PEG-12, either alone or as one of the lipids in a mixture. The method may further comprise providing an active compound; and combining the active compound with the lipid solution and the aqueous solution.

In another aspect, the invention includes a liposome suspension. The suspension comprises one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The suspension may comprise a single lipid. The lipid may comprise dioleolylglycerol-PEG-12. The suspension may further comprise an active compound, which may be selected from the group described above.

In another aspect, the invention includes a composition for combining with an aqueous solution to form a liposome suspension. The composition comprises one or more lipids, where the lipids as an aggregate have a Pₐ between about 0.84 and 0.88, a Pᵥ, between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The composition may comprise a single lipid. The composition may comprise dioleolylglycerol-PEG 12. The composition may further comprise an active compound selected from the group above. The composition may be provided in a sealed container, where the container also contains an inert gas to prevent oxidative degradation.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise, a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops.

The compositions may be administered topically, inter-orally, vaginally or rectally.

PEG-12 Glyceryl Dioleate was obtained from Global 7 (New Jersey) for the following formulations. This can be substituted for the lecithin w/w % as needed to accomplish the formulation, or applied as set forth below.

In the following formulations, the “set percentage” w/w % of reduced glutathione is selected from 3.3%, 4%, 5%, 6%, 7%, 7.5%, 8%, 8.5% or 9% or amounts approximately to those percentages. Also may be selected 8.25% w/w. The amount of 3.3% w/w Glutathione (reduced) is 123 mM in concentration in the liposomes.

### EXAMPLE 3A

Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water , the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

### EXAMPLE 3B

Spontaneous Liposomes for Intravenously Administered Therapeutic Compound and as a Drug Solubilization Vehicle for Use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w. Deionized water is added and gently mixed.

Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered. These attributes allow a secondary advantage of the invention by the QuSome liposome encapsulating a radionuclide useful for targeting tumors with either diagnostic radionuclides or therapeutic radionuclides. The QuSome self-forming liposome is of such as size and the presence of the steric stability with PEG results in long circulation time and an increased accumulation in the fine trabecular mesh of blood vessels supplying growing tumors. This characteristic allows for improved diagnostics as more radionuclide accumulates around the tumor improving the image of scans. This characteristic of accumulating in the trabecular mesh of blood vessels leading to tumors also leads to an improved therapeutic. The accumulation of QuSome self-forming liposomes in the blood vessel supply to tumors increases the radiation dosing to this area, creating damage to the tumor blood vessels creating an anti-angiogenic effect, resulting in a decreased supply of blood to the tumor and leading to death of tumor cells.

The concentration of liposomal glutathione in the Qusome formulation is 5% for topical application. It is possible to use the Qusome technology in creating an oral formulation also and the 8.25% glutathione in w/w concentration may be used in the oral formulation.

3-Bromopyruvate Dosages for Liver Cancers:

Transcatheter intra-arterial Bolus infusion of 250 mg 3-bromopyruvate either as a bolus or drip. After 20 minutes an infusion of liposomal reduced glutathione (LRG) 2000 mg may be infused and/or an oral ingestion of 2500 mg of liposomal reduced glutathione may be ingested orally. The oral liposomal reduced glutathione dose may be repeated every 8 hours in the next 24 hour period. Subsequent doses of 2000 mg twice a day of liposomal reduced glutathione are administered daily for 7 days following the administration of 3BP to minimize the effects of tumorlysis debris known as tumorlysis syndrome. Analogs included in the reference “3BP” include Bromopyruvic acid and 3-BrOP (3-bromo-2-oxopropionate-1-propyl ester).

For lung tumors 3-bromopyruvate 250 mg is administered by inhalation of nebulization of 250 mg dissolved in normal saline. This dose is then followed 20 minutes later by nebulization of plain glutathione 2000 mg or in the preferred mode liposomal reduced glutathione 2000 mg nebulized and/or 2500 mg liposomal reduced glutathione ingested orally with repeat doses every 8 hours for 3 additional doses. Subsequent doses of 2000 mg twice a day of liposomal reduced glutathione are administered daily for 7 days following the administration of 3BP to minimize the effects of tumorlysis debris known as tumor lysis syndrome.

Broader BP Method to Treat Cancers

The addition of 3-bromopyruvate will extend the tumorcidal activity to a wide range of cancers. The timing of the administration of 3BP and liposomal glutathione is critical as liposomal reduced glutathione (LRG) will neutralize the activity of 3BP. Thus for use in hepatic tumors, 3BP can be administered by intra-arterial infusion. About 15 minutes after the infusion of 3BP liposomal glutathione can be infused intravenously (2000 mg intravenously) and another 2500 (approximately 6 teaspoons of liposomal reduced glutathione) can be ingested orally. The dose of oral liposomal reduced glutathione 2500 can be repeated every 8 hours for the next 24 hours to decrease the side effects of the 3BP and to facilitate the removal of the cell debris that will be liberated from killed cancer cells.

### REFERENCES

1. Baay M, Brouwer A, Pauwels P, Peeters M, Lardon F. Tumor cells and tumor-associated macrophages: secreted proteins as potential targets for therapy. Clin Dev Immunol. 2011;2011:565187. PMCID: 3227419.

2. Ohtaki Y, Ishii G, Nagai K, Ashimine S, Kuwata T, Hishida T, et al. Stromal macrophage expressing CD204 is associated with tumor aggressiveness in lung adenocarcinoma. J Thorac Oncol. 2010;5(10):1507-15. PMID 20802348.

3. Espinosa I, Jose Carnicer M, Catasus L, Canet B, D'Angelo E, Zannoni G F, et al. Myometrial invasion and lymph node metastasis in endometrioid carcinomas: tumor-associated macrophages, microvessel density, and HIF1A have a crucial role. Am J Surg Pathol. 2010;34(11):1708-14. PMID 20962622.

4. Kurahara H, Shinchi H, Mataki Y, Maemura K, Noma H, Kubo F, et al. Significance of M2-polarized tumor-associated macrophage in pancreatic cancer. J Surg Res. 2011;167(2):e211-9. PMID 19765725.
5. Fabriek B O, van Bruggen R, Deng D M, Ligtenberg A J, Nazmi K, Schornagel K, et al. The macrophage scavenger receptor CD163 functions as an innate immune sensor for bacteria. Blood. 2009;113(4):887-92. PMID 18849484

6. Sachdev R, Robbins J, Kohler S, Vanchinathan V, Schwartz E J, Sundram U N. CD163 expression is present in cutaneous histiocytomas but not in atypical fibroxanthomas. Am J Clin Pathol. 2010;133(6):915-21. PMID 20472850.

7. Jensen T O, Schmidt H, Moller H J, Hoyer M, Maniecki M B, Sjoegren P, et al. Macrophage markers in serum and tumor have prognostic impact in American Joint Committee on Cancer stage I/II melanoma. J Clin Oncol. 2009;27(20):3330-7. PMID 19528371.

8. Shabo I, Stal O, Olsson H, Dore S, Svanvik J. Breast cancer expression of CD163, a macrophage scavenger receptor, is related to early distant recurrence and reduced patient survival. Int J Cancer. 2008;123(4):780-6. PMID 18506688.

9. Shabo I, Olsson H, Sun X F, Svanvik J. Expression of the macrophage antigen CD163 in rectal cancer cells is associated with early local recurrence and reduced survival time. Int J Cancer. 2009;125(8):1826-31. PMID 19582880.

10. Kamper P, Bendix K, Hamilton-Dutoit S, Honore B, Nyengaard J R, d'Amore F. Tumor-infiltrating macrophages correlate with adverse prognosis and Epstein-Ban virus status in classical Hodgkin's lymphoma. Haematologica. 2011;96(2):269-76. PMCID: 3031695.

11. Fujii N, Shomori K, Shiomi T, Nakabayashi M, Takeda C, Ryoke K, et al. Cancer-associated fibroblasts and CD163-positive macrophages in oral squamous cell carcinoma: their clinicopathological and prognostic significance. J Oral Pathol Med. 2012. PMID 22296275.

12. Sihto H, Bohling T, Kavola H, Koljonen V, Salmi M, Jalkanen S, et al. Tumor Infiltrating Immune Cells And Outcome of Merkel Cell Carcinoma: A Population-based Study. Clin Cancer Res. 2012. PMID 22467679.

13. Soult M C, Feliberti E C, Silverberg M L, Perry R R. Merkel cell carcinoma: High recurrence rate despite aggressive treatment. J Surg Res. 2012. PMID 22537840.

14. Werchau S, Toberer F, Enk A, Dammann R, Helmbold P. Merkel cell carcinoma induces lymphatic microvessel formation. J Am Acad Dermatol. 2011. PMID 22050913.

15. Fields R C, Busam K J, Chou J F, Panageas K S, Pulitzer M P, Kraus D H, et al. Recurrence and survival in patients undergoing sentinel lymph node biopsy for merkel cell carcinoma: analysis of 153 patients from a single institution. Ann Surg Oncol. 2011;18(9):2529-37. PMID 21431988.

16. Levitskaia T G, Morris J E, Creim J A, Woodstock A D, Luders T, Curry T L, et al. Aminothiol receptors for decorporation of intravenously administered (60)Co in the rat. Health Phys. 2010;98(1):53-60. PMC2818207.

17. Peterson J D, Herzenberg L A, Vasquez K, Waltenbaugh C. Glutathione levels in antigen-presenting cells modulate Th1 versus Th2 response patterns. Proc Natl Acad Sci U S A. 1998;95(6):3071-6. PMID 9501217

I claim:

1. An anti-cancer pharmaceutical composition enabling delivery after oral administration of a therapeutically effective amount of glutathione (reduced) and 3-BP comprising:
reduced glutathione stabilized in a liposomal pharmaceutical carrier capable of being ingested orally, and capable of delivering glutathione (reduced) in a physiologically active state to improve symptoms in disease states by transfer of the glutathione into animal cells,
where the concentration of reduced glutathione in the entrapped aqueous space of the liposomes is 8.25% w/w; and
3-BP.

2. An anti-cancer pharmaceutical composition comprising:
reduced glutathione stabilized in a liposomal pharmaceutical carrier capable of being ingested orally, and capable of delivering glutathione (reduced) in a physiologically active state to improve symptoms in disease states by transfer of the glutathione into animal cells,
where the concentration of reduced glutathione in the entrapped aqueous space of the liposomes is at least 123 mM; and
3-BP.

3. A method of treating Merkle cell carcinoma, comprising the following step:
administering reduced glutathione stabilized in a liposomal pharmaceutical carrier capable of being ingested orally, and capable of delivering glutathione (reduced) in a physiologically active state to improve symptoms in disease states by transfer of the glutathione into animal cells,
where the concentration of reduced glutathione in the entrapped aqueous space of the liposomes is 8.25% w/w.

4. The method of treating Merkle cell carcinoma according to claim 3, further comprising the following step:
administering 3-BP.

5. A method of treating Merkle cell carcinoma, comprising the following steps:
administering reduced glutathione stabilized in a liposomal pharmaceutical carrier capable of being ingested orally, and capable of delivering glutathione (reduced) in a physiologically active state to improve symptoms in disease states by transfer of the glutathione into animal cells,
where the concentration of reduced glutathione in the entrapped aqueous space of the liposomes is at least 123 mM; and
administering 3-BP.
